(19) United States
(12) Patent Application Publication
(10) Pub. No.: US 2014/0336634 A1
(43) Pub. Date: Nov. 13, 2014

(54) MULTIFUNCTIONAL ATTACHMENT FOR ELECTROCAUTERY SURGICAL DEVICE

(71) Applicant: Ricardo Alexander Gomez, Lighthouse Point, FL (US)

(72) Inventor: Ricardo Alexander Gomez, Lighthouse Point, FL (US)

(73) Assignee: NEW WAVE SURGICAL CORP., Pompano Beach, FL (US)

(21) Appl. No.: 14/275,024

(22) Filed: May 12, 2014

Related U.S. Application Data
(60) Provisional application No. 61/822,760, filed on May 13, 2013.

(51) Int. Cl. A61B 18/04

(52) U.S. Cl. CPC A61B 18/04; A61B 2018/00595
USPC 606/40

(57) ABSTRACT

A multifunctional surgical device particularly for use during electro cauterization for irrigation, and/or smoke and/or fluid evacuation as required. The device comprises a working hub incorporating an elongated tube and including a vacuum port disposed through an exterior wall of the hub, the port being fluidly connectable to a source of vacuum or irrigation fluid; a stopcock which selectively connects the vacuum port and an outlet into the stopcock from the elongated tube, the stopcock selectively allowing a fluid connection to be formed between the vacuum port and the elongated tube; an open central passage longitudinally disposed through the device; the elongated tube having proximal and distal ends and being disposed co-linearly with the central passage, sized and configured to seat within a cannula and receive within the central passage the shaft of an endoscopic tool, while space remains therearound to pass fluid in either direction about the tool shaft.

### CROSS REFERENCE TO RELATED APPLICATION

This non-provisional utility patent application claims priority from provisional patent application Ser. No. 61/822,760 filed on May 13, 2013 and entitled Multifunctional Attachment for Electro-Cautery Surgical Devices, the teachings of which are incorporated herein by reference.

### FIELD OF THE INVENTION

This invention is related to laparoscopic instruments. More particularly, this invention is a multifunction vacuum attachment device for a standard electrocautery device.

### BACKGROUND OF THE INVENTION

Originally, surgeries were performed through large incisions in the body. These open surgeries and their large incisions allowed surgeons to visually see and manipulate the diseased part of the body with their hands. Recently, minimally invasive surgery has become popular because of the benefits associated with smaller incisions. Termed minimally invasive surgery or keyhole surgery, these surgeries utilize ports called cannulas inserted through the abdomen. They pass through the abdominal wall by piercing through the layers of skin. The puncture wounds created are small and lessen postoperative healing time and pain. There are a number of drawbacks for the surgeon in minimally invasive surgery. Of most concern, is the surgeon's loss of direct visual and tactile contact with the patient's internal organs. In order for the surgeon to visualize them, one cannula port contains a laparoscopic camera for visualization. The image generated by the camera is sent to a viewing screen that displays the inner body parts. The trend towards smaller sized cannulas because of patient benefits creates challenges for the manufacturers of minimally invasive medical devices. The small sizes create extreme dimensional requirements. The devices often must be thin but remain strong. The conflicting requirements require special materials and novel engineering design.

Another trend in surgery is increased awareness of health and safety of the staff performing the procedures. One area of concern is surgical smoke created by electrocautery devices and electrosurgical devices during minimally invasive surgical procedures.

Electrocautery is a term used to refer to both a surgical instrument and the procedure for which that instrument is used. An electrocautery instrument or apparatus is a surgical tool used for tissue dissection and/or hemostasis. In an electrocautery surgical procedure, the working end of an electrocautery device is heated by a high voltage, high frequency alternating current passed through an electrode in the device. The now heated working portion of the electrocautery instrument is used for the destruction of tissue, such as for removing warts or polyps and cauterizing small blood vessels to limit blood loss during surgery. There are two types of electrocautery: bipolar and monopolar.

In bipolar electrocautery, active and return electrodes are incorporated into a single handheld electrocautery instrument, so that the current passes between the tips of the two electrodes and affects only a small amount of tissue. In monopolar electrocautery current is applied through a handheld active electrode and travels back to the generator through an inactive electrode attached to the patient (the grounding pad), so that the patient is part of the electrical circuit.

Electrosurgery is the application of a high frequency electric current to biological tissue as a means to cut, coagulate, desiccate, or fulgurate tissue. This is accomplished by converting electrical energy into heat through tissue resistance to the passage of the electrical current. Two types of current are utilized in electrosurgery, damped and undamped; a damped current destroys and coagulates tissue and stops bleeding, and undamped current destroys minimal tissue and incises tissue.

Electrocautery devices are used by surgeons to sear and cut tissue during a surgery. In the surgical process, body tissue can char and produce smoke or steam byproducts.

Electrocautery shall be more frequently discussed herein. However, those of ordinary skill in the art will appreciate that the invention disclosed and claimed herein is equally useful with other electrosurgical devices and methods as well.

Another trend in surgery is an increased number of robotic surgeries. Robotic surgeries have recently become more prevalent for performing laparoscopic surgeries. However, robotic surgeries create additional problems for smoke evacuation. Robotic surgeries require a surgeon to operate from a console that is separated from the patient. During a robotic surgery, assistants must add and remove laparoscopic tools for the surgeon. Exchanging laparoscopic tools, including electrocautery devices is a lengthy procedure because the tools often require disconnection from the robotic system. Additionally, robotic surgeries require smaller trocar such as the 8.5 mm and 10 mm. Many of the current multifunction electrocautery devices are not designed for these small trocars. There is a need for an improvement of smoke evacuation systems for robotic surgery.

All electrocautery devices create smoke. Smoke created during laparoscopic procedures can be difficult to remove because the enclosed abdomen in laparoscopic procedures contains the surgical smoke within the patient's body. The retained smoke, if not evacuated from the body, interferes with the surgeon's visualization of the procedure. The lack of visibility can lengthen the procedure, adding to costly operating room time and subjecting the patient to increased time under anesthesia. Other studies have indicated that surgical smoke is potentially infectious and toxic. Many hospitals are increasingly emphasizing safety and have demanded devices that will remove smoke from the operating room without side effects to the patient or surgical team.

During surgery there is a need to wash away blood after coagulating (burning an area of tissue) to assure the vessel is no longer bleeding. Presently, surgeons have to remove the instrument, insert a suction device, and then reinsert the coagulator again. A need exists for a device that allows those functions to occur without removing the coagulator from the cannula by sliding the multifunction laparoscopic device through the cannula.

Improvements to electrocautery devices have included smoke evacuators integrated with the electrocautery device. The addition of a smoke evacuator to the electrocautery device creates a multifunction tool for surgery. The smoke evacuator is often located near the tip of the electrocautery device so that the smoke can be quickly evacuated after it is produced. Some electrocautery devices have been further enhanced by adding irrigating capabilities. The irrigating capabilities allow the surgeon to wash blood or tissues away from the cutting tip of the electrocautery. These multifunction devices can perform suction (smoke evacuation), irrigation, and electrocautery. The combination of multiple features into one device reduces the number of devices needed for a surgical procedure. Multifunction devices reduce operating time. However, these devices do not allow the surgeon to operate with an electrosurgical device. Instead the surgeon must learn how to operate a new electrocautery device incorporated into their multifunctional unit.

Stand alone smoke evacuation devices and methods have been developed for laparoscopic procedures utilizing small trocar ports. The most popular method adopted by surgeons has been the opening of the trocar insufflation port. By opening a path from the pressurized abdomen to the surgical room, the opening of the insufflation port forces the smoke retained within the abdomen to the operating room. The opening of the trocar insufflation port has been widely adopted by surgeons because of its simplicity. It does not require an additional device and can be operated in an expedient manner. The downsides to the opening of the trocar insufflation port are the safety concerns associated with surgical smoke and the decrease in abdominal insufflation pressure when CO₂ is released from the body.

Because of the disadvantages associated with the trocar insufflation port method, devices that attach to trocar ports have been developed. These devices act as filters, removing smoke from the CO₂ within the body and recirculating the smoke free CO₂ back into the body. Generally, the filter devices connect to multiple trocar ports. The CO₂ from the body is diverted through the filter because of pressure differences created by the pressurized abdomen. One advantage of the system is that insufflation pressure of the abdomen can be maintained while smoke is removed. However, it has been found that these devices do not rapidly reduce the smoke within the body. The distance from the source of the surgical smoke to the trocar cannula opening within the body and the smoke evacuation filter causes slow smoke evacuation.

What is needed in the art, however, is an integrated device that can provide suction, irrigation and smoke evacuation while attaching to the surgeon's preferred electrocautery device.

### SUMMARY OF THE INVENTION

The present invention is a multifunction laparoscopic device that attaches to standard electrocautery or laparoscopic tools to provide smoke evacuation, irrigation, and suction of bodily fluids. Advantageously, the multifunction device of the present invention provides integrated suction, irrigation, and smoke evacuation functionality, such that the electrocoagulator need not be removed from a patient's body and the surgeon can continue to use a favorite electrocoagulator. The multifunction laparoscopic device comprises an elongated tube and a vacuum port. The vacuum port comprises housing with an integral passage for smoke and other fluids. The vacuum port is adapted to receive a sealing mechanism. The multifunction laparoscopic device is adjustable along the length of an electrocautery or laparoscopic tool cauterizing shaft. The adjustment of the multifunction laparoscopic device allows smoke evacuation and the intra abdominal suction of fluids. The multifunction laparoscopic device maintains insufflation pressure inside the patient's body during the movement of the device along the cauterizing shafts of standard electrocautery devices because of the sealing mechanism. The vacuum port further comprises a port for removing smoke or other fluids during an operation.

The present invention is uniquely able to suction smoke and bodily fluids through the annular opening between the shaft of the electrocautery device and the trocar cannula housing without removing the electrocoagulator from the patient's body. The multifunction attachment is disposed coaxially around the shaft of an electrocautery device or a laparoscopic tool. The invention further improves upon previous devices by allowing electrocautery, smoke evacuation, irrigation, and suction through a single laparoscopic trocar port.

Another advancement of the current invention is the small size of the multifunction laparoscopic device. The small size of the device reduces the affect the attachment has on the functionality of current laparoscopic devices including trocars and electrocautery instruments.

The multifunction laparoscopic device is sized for insertion into standard size trocars/cannulas without interference with the cannula inner diameter. The extremely thin wall construction of the multifunction laparoscopic device's elongated tube is an advance in medical irrigator and smoke evacuator design. By eliminating the dimensional interference between the trocar cannula housing and the outer surface of the multifunction laparoscopic device, standard electrocautery devices, when combined with the multifunction laparoscopic device, will fit through standard sized trocars/cannulas and can be operated as currently practiced by surgeons.

The multifunction laparoscopic device vacuum port, seals to the outer diameters of standard electrocautery device shafts by a sealing mechanism within the evacuation port housing. The sealing mechanism closes against the shaft of the electrocautery device to prevent loss of insufflation pressure during surgery. When the electrocautery device is cauterizing bodily tissue, the valve of the vacuum port housing is opened for smoke evacuation. The opening of the valve provides a path for surgical smoke to exit the abdomen.

The multifunction laparoscopic device can be positioned anywhere along the proximal end of the shaft of the electrocautery device. Since the position of the multifunction laparoscopic device is adjustable, the smoke evacuation ability of the device is enhanced because the distal end of the elongated tube can be positioned close to the origin of smoke generation. The distal end of the elongated tube can be positioned past the tip of the electrocautery device. The positioning of the elongated tube past the electrocautery tip increases the amount of fluid that can be suctioned by the suction functionality of the multifunction laparoscopic device. Suctioning of fluids can be performed by adjusting the multifunction laparoscopic device's location on the electrocautery shaft. The sealing mechanism of the multifunction device maintains insufflation pressure while adjusting the multifunction laparoscopic device. The sealing mechanism is an elastomeric material that seals around the shaft of the electrocautery device to maintain insufflation pressure. Any body fluids are transported through the same path as the surgical smoke.

In one embodiment, the vacuum port of the multifunction laparoscopic device attaches to a standard vacuum source with specialized tubing. The specialized tubing is easily extensible so that the surgeon's range of motion will not be affected during the operation. The specialized tubing has a spring or “slinky” configuration for enhanced flexibility.

The vacuum port housing has a small overall thickness to maximize the surgeon's range of motion. The position of the vacuum port housing between the trocar and the body of the electrocautery device necessitates its small size. During a surgery, repositioning of the electrocautery device occurs frequently. The surgeon continuously moves the electrocautery device into and out of the body of the patient in order to position the tip of the electrocautery device near the target tissue. These motions decrease the distance between the trocar and the distal surface of the vacuum port housing of the electrocautery device. Since the multifunction laparoscopic device is attached to the shaft of the electrocautery device below the electrocautery device's housing, the effective length of the combined attachment and the electrocautery device is reduced when compared with the electrocautery device by itself. The depth within the body that the electrocautery device can achieve is reduced by the thickness of the multifunction laparoscopic device's vacuum port housing.

Another embodiment of the multifunction laparoscopic device provides tip attachments for the end of the elongated tube. The elongated tube can receive attachments such as absorptive surgical peanuts, specialized irrigation heads, specialized suction heads, dissectors, standard instrument heads (i.e.: scissors, blades, clamps, etc.), heads for dispensing active agents (i.e. hemostatic agents, antibacterial agents, etc.) or other attachments that are typically used in laparoscopy and would enhance the capabilities of the multifunction laparoscopic device.

One of several advantages of the present invention, is that it provides a tool that allows surgeons to remove blood from the surgical site after coagulating (burning an area of tissue) to assure the vessel is no longer bleeding, without requiring that the surgeon to remove the surgical instrument, insert a suction device, and reinsert the coagulator again. The present invention does not require that any instruments be brought in or out of the body to perform those functions.

These and other aspects, features, and advantages of the present invention will become more readily apparent from the attached drawings and the detailed description of the preferred embodiments, which follow.

### BRIEF DESCRIPTION OF THE DRAWINGS

The preferred embodiments of the invention will hereinafter be described in conjunction with the appended drawings provided to illustrate and not to limit the invention, in which:

FIG. 1 presents a front isometric view of a multifunctional device according to the present invention;

FIG. 2 presents a rear isometric view of the device of FIG. 1;

FIG. 3 presents an exploded isometric view of the device of FIG. 1;

FIG. 4 presents a cross sectioned elevation view of the multifunctional device of FIG. 1, the section being taken along section line 4-4 of FIG. 1;

FIG. 5 presents a cross sectioned elevation view of the multifunctional device of FIG. 1, introducing a retaining clip, the section being taken along section line 5-5 of FIG. 1;

FIG. 6 presents a top isometric view of a working hub of the multifunctional device of FIG. 1;

FIG. 7 presents a bottom isometric view of the working hub of the multifunctional device of FIG. 1;

FIG. 8 presents a top isometric view of the top portion of the retaining clip for use with the multifunctional device of FIG. 1;

FIG. 9 presents a bottom isometric view of the top portion of a retaining clip for use with the multifunctional device of FIG. 1;

FIG. 10 presents a sectioned end view of a retaining clip for use with the multifunctional device of FIG. 1, the section being taken along section line 10-10 of FIG. 8;

FIG. 11 presents a magnified, front, isometric view of the stopcock for the multifunctional device of FIG. 1;

FIG. 12 presents a top, isometric view of an alternative embodiment of a retaining clip;

FIG. 13 presents a sectional end view of the retaining clip of FIG. 12, the section being taken along section line 13-13 of FIG. 12; and

FIG. 14 presents a side assembly view of the present invention in use during laparoscopic surgery.

Like reference numerals refer to like parts throughout the several views of the drawings.

### DETAILED DESCRIPTION

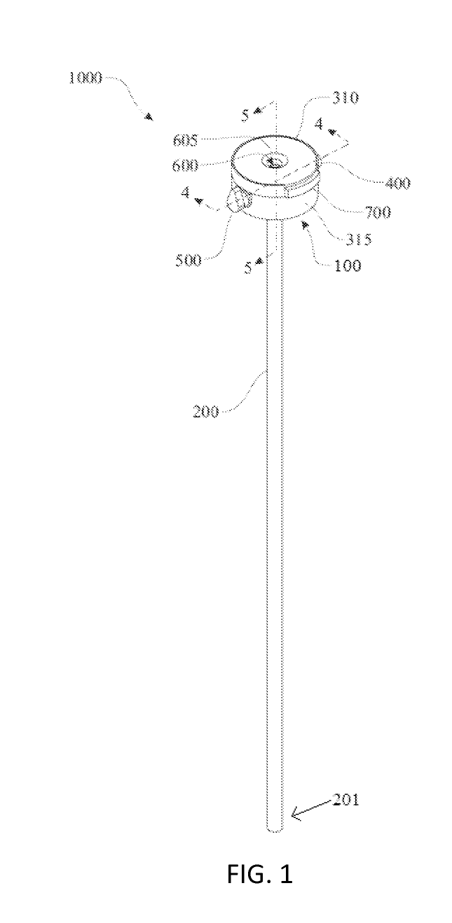

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure. For purposes of description herein, the terms “upper”, “lower”, “left”, “rear”, “right”, “front”, “vertical”, “horizontal”, and derivatives thereof shall relate to the invention as oriented in FIG. 1.

Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

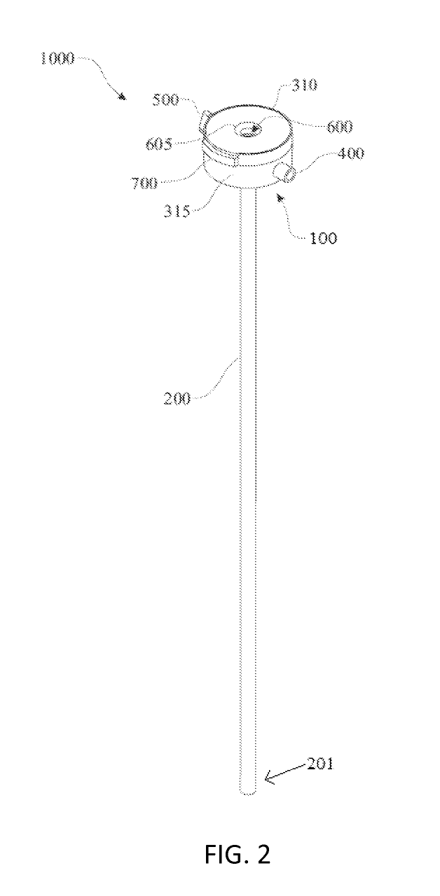

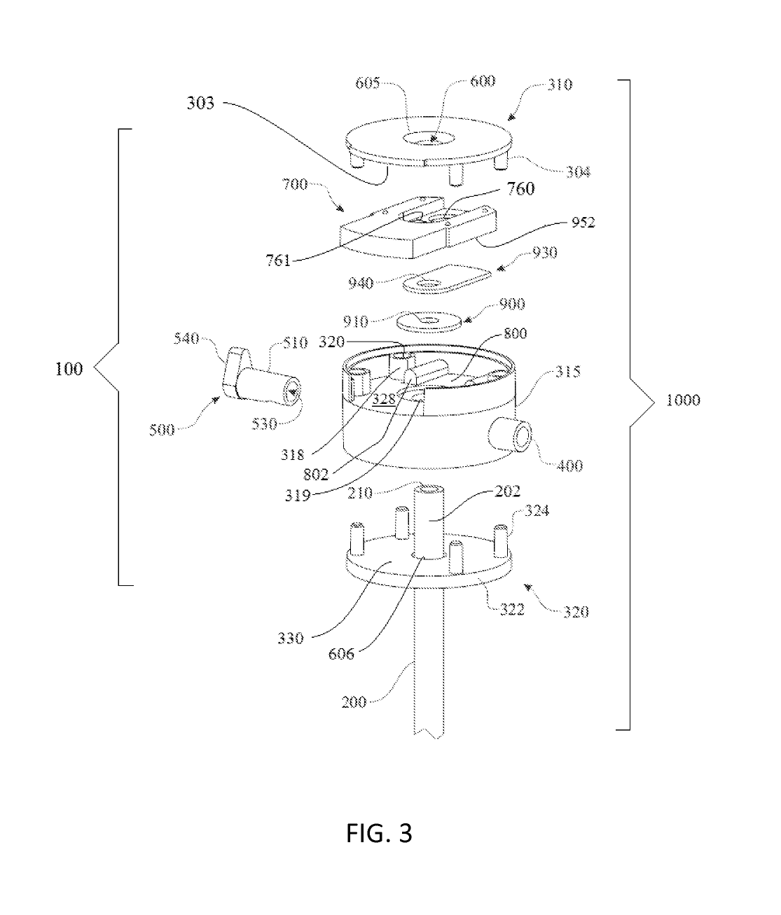

A front isometric view of a multifunctional, laparoscopic, device 1000 is illustrated in FIG. 1. A rear isometric view of the multifunction laparoscopic device 1000 is illustrated in FIG. 2. The multifunction laparoscopic device or tool 1000 generally comprises: a working hub 100 in the form of a vacuum attachment 300 of FIG. 3 and an elongated hollow tube 200 extending distally therefrom. The elongated hollow tube 200 is preferably constructed of a strong, lightweight, biocompatible material suitable for use during surgery, preferably of plastic, aluminum, stainless steel, or other rigid metal. The elongated hollow tube 200 can have an extremely thin wall thickness, as thin as 0.004″. When the elongated hollow tube 200 is constructed of metal, preferably the elongated hollow tube 200 is coated with a nonconductive material such as parylene (not shown). The parylene coating of the metal tube prevents the elongated hollow tube 200 from conducting electricity.

The working hub 100 is seen to be in the form of a generally wide but somewhat flattened cylinder. However, those of skill in the art will appreciate that the working hub 100 may have any convenient or desired shape. The working hub 100 includes a substantially flat proximal surface 310 and a substantially parallel substantially flat distal surface 320 (FIG. 3), with a cylindrical outer or side wall 315 (FIG. 2), extending between the proximal surface 310 and distal surface 320.

The working hub 100 further includes a stopcock 500, which extends through the side wall 315 of working hub 100, as will better be described below. The working hub 100 further includes a slot 800 (FIG. 3) for receiving a retaining clip 700, as will also be better described below.

The working hub 100 further includes an outwardly extending vacuum port 400, which traverses the cylindrical side wall 315 at a location substantially opposite to the location of stopcock 500.

The working hub 100 also includes a central hollow passage 600 which is coaxial with a central axis of working hub 100 and communicates with openings in both the proximal, generally flat wall 310 and the distal, generally flat wall 320, as best illustrated in FIG. 3. In a preferred embodiment, the central passage 600 comprises a funnel or conical shaped proximal opening 605 in the proximal wall 310 and an opening 606 in the distal wall 320 of FIG. 3.

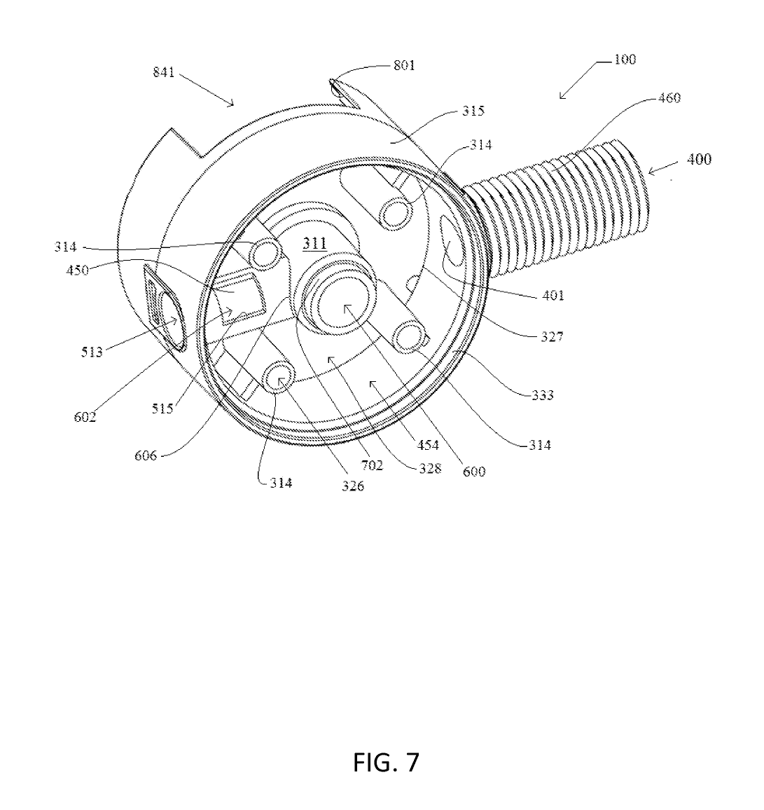

An exploded, isometric view of the multifunctional laparoscopic device 1000 is illustrated in FIG. 3. The distal wall 320 of working hub 100 is in the form of a flat plate of FIG. 3 and has an inner proximal surface 330 from which bottom posts 324 extend upwardly into the hub 100 for engaging within post bottom receiving openings 326 in posts 314 of FIG. 7 extending downwardly from a distal surface 327 of a center wall 328 of the hub 100 (FIG. 7). The proximal wall 310, also in the form of a flat plate, includes a distal surface 303 from which posts 304 extend downwardly for engaging with post receiving members 318 defining receiving openings 310 formed in a proximal surface 319 of center wall 328. The flat proximal and distal walls 310 and 320, respectively, form an enclosed working hub 100 of FIG. 1. A peripheral edge 322 of the distal wall 320 of FIG. 3 seals against an inner surface undercut flange 333 of the peripheral wall 315 of the working hub 100 (FIG. 7).

Turning back to FIG. 3, the working hub 100 is also provided with a seal or gasket 900 having a central bore 910 which is coaxial with the central passage 600 when seated appropriately upon proximal surface 319 of the center wall 328. The seal 900 may be formed from any reasonably flexible material suitable for preventing leakage of bodily fluids therethrough or therearound, the seal 900 preferably being made of a rubber, or plastic polymer of any known composition. A retaining clip 700 is received in a slot 800 of the working hub 100 and engages therebeneath a second seal member 930 with a center bore 940, along a distal surface 952 thereof. Center bore 940 of second seal member 930 is also coaxial with central passage 600, which passes through the center wall 328 when the clip 700 is appropriately seated and the second seal member is properly oriented to seat upon first seal member 900. Second seal member 930 is also preferably made of a rubber or plastic polymer of any suitable known composition.

The elongated hollow tube 200 engages the central passage 600 of the working hub 100 and, subsequently, the central bore 910 of the seal 900. A bore 210 extends through the length of elongated hollow tube 200. In one preferred embodiment, the central passage 600 has a larger diameter than the bore 210 of elongated tube 200, though this should not be construed as limiting.

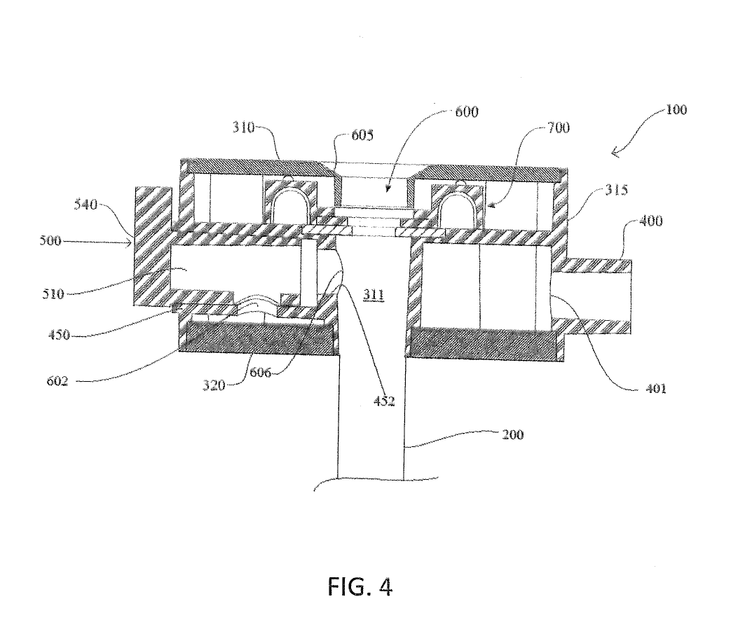

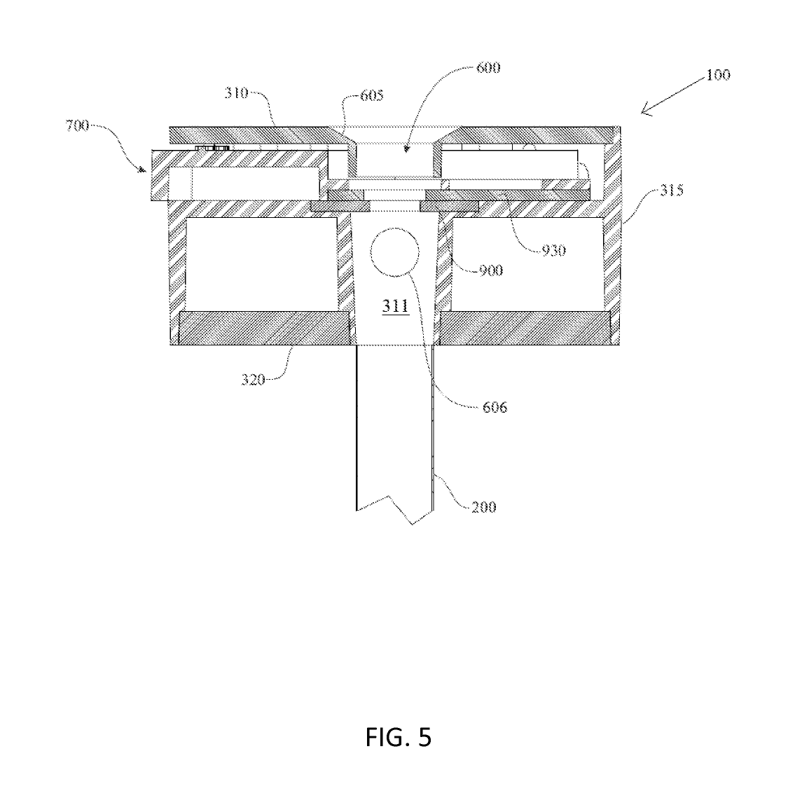

Cross sectional views of multifunctional laparoscopic device 1000 are illustrated in FIGS. 4 and 5. The illustrations present relationships between the central passage 600, the elongated tube 200, the retaining clip 700, and the retaining clip seal member 930 of FIG. 3, the vacuum attachment seal member 900, and the stopcock 500. As presented in FIG. 5, the retaining clip seal member 930 seats against the seal member 900 of FIG. 3 and the central passage 600 is shown to extend through the multifunctional laparoscopic device 1000 when seal member 930 is appropriately positioned, as shown.

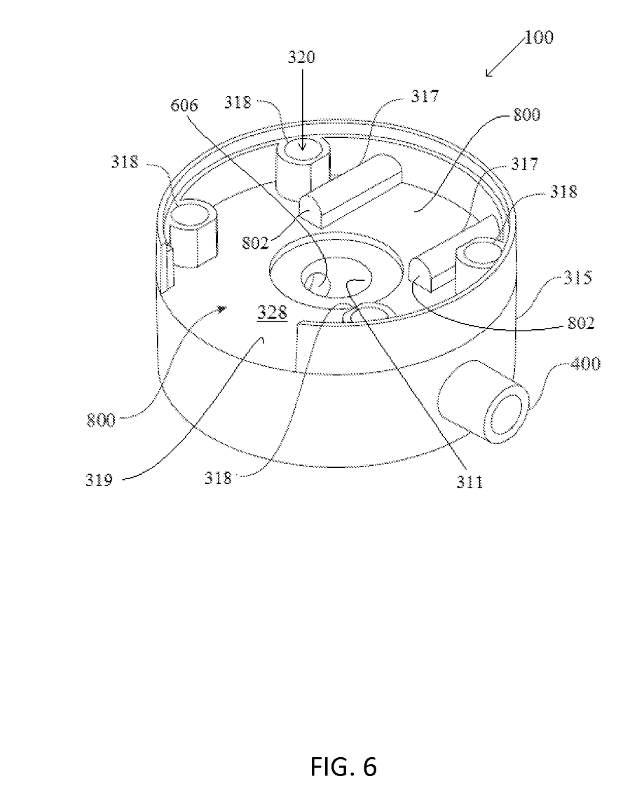

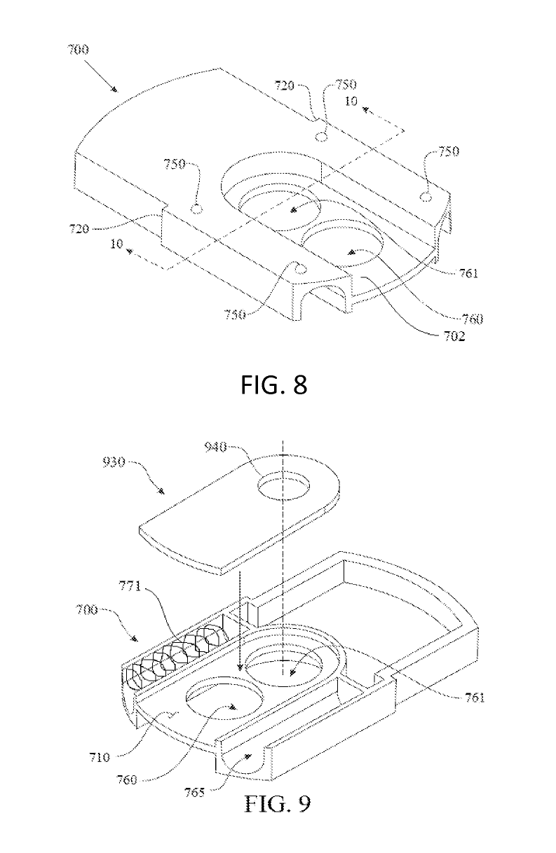

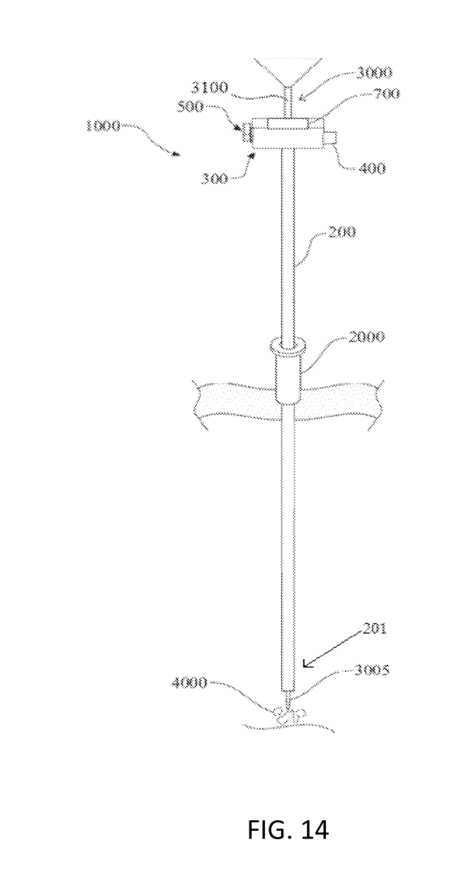

In operation, when the retaining clip 700 is fully engaged within the slot 800 of the working hub 100, the opening 940 through the retaining clip seal member 930 is aligned with the central passage 600 of the multifunction laparoscopic device 1000. In this configuration, both seal members 900 and 930 of the multifunction laparoscopic device 1000 seal against a shaft of an electrocautery device or laparoscopic tool 3000 (FIG. 14) extending through the multifunction laparoscopic device 1000. The retaining clip 700 when being partially slid radially outwardly within the retaining clip slot 800 of FIG. 6 prevents the loss of insufflation pressure when the laparoscopic tool 3000 is removed from central passage 600. The retaining clip 700 may also be slid out of retaining clip receiving slot 800 as a result of a biasing force generated by biasing members or springs 770 seated in recesses 765 (FIG. 9) of the retaining clip 700. These biasing members or springs 770 are compressed against inner ends 802 of the retaining clip guides 317, as illustrated in FIG. 6 maintaining the retaining clip 700, when the retaining clip 700 is fully inserted into the working hub 100. As the retaining clip 700 slides out of the retaining clip slot 800 through an opening 841 in cylindrical wall 315, the opening 940 of FIG. 1 in the seal member 930 of the retaining clip 700 and the central passage 600 are no longer aligned. In this skewed position, the two seal members 900 and 930 of the multifunction laparoscopic device 1000 prevent the passage of carbon dioxide from the abdomen. The retaining clip 700 is maintained within the working hub 100 by contact between the retaining clip shoulders 720 (FIG. 8) and the edges of the opening 841 in cylindrical outer wall 315.

A detailed top view into the working hub 100 showing the proximal surface 319 of center wall 328 is illustrated in FIG. 6 wherein hollow post receiving means 318 including openings 320 for receiving posts 304 FIG. 3 of the proximal wall 310 of the working hub 100 are provided. The generally cylindrical outer wall 315 of working hub 100 above the level of the center wall 328 also defines retaining clip slot 800, and retaining clip guides 317.

A detailed bottom view the distal surface 327 of the center wall 328 of the working hub 100 is illustrated in FIG. 7 and shows the hollow post attachment means 314 FIG. 1 including openings 326 FIG. 7 to which the posts 324 on the proximal surface 330 of the bottom wall 320 are engaged. Also visualized is a nipple like structure or center tube 311 defining a distal connector for tube 311 onto which a proximal end 202 of the elongated hollow tube 200 of FIG. 3 is fed and frictionally engaged. Vacuum port 400 is shown to be disposed through the sidewall 315 of the working hub 100. Further, the working hub 100 is seen to include a port 602 of FIG. 7 in a stopcock sleeve 450 FIG. 4 thereof which selectively aligns with a cooperating side wall port or opening 520 in the stopcock 500 of FIG. 3 to provide for control for the flow of smoke 4000, fluids, and the like through the multifunction laparoscopic device 1000.

The longitudinal bore 520 FIG. 11 of the stopcock 500 extends into the working hub 100 FIG. 7 to a point where it intersects with a port 606 feeding into central opening 600 within the center tube 311 of FIG. 6.

A top isometric view of the top of retaining clip 700 is illustrated in FIG. 8. The retaining clip 700 fits into the slot 800 as shown in FIG. 6. The retaining clip 700 is releasably inserted into the slot 800 FIG. 6 and may include quick connect features, such as shoulders 720 for cooperatively, releasably engaging against side walls 801 FIG. 7 of the retaining clip receiving slot 800 FIG. 6. Two openings an outer opening 760 and an inner opening 761 extend through a distal stepped down portion 702 of the retaining clip 700, with each opening serving a specialized function related to retaining second seal clip member 930 FIG. 9. In this respect, opening 761 aligns with the centering 940 in the second seal member 930 FIG. 1 to allow passage of a laparoscopic tool through central passage 600 FIG. 1 while the outer opening 760 allows the second seal member 930 FIG. 3 to deform, creating a closure contact between a solid area of second seal member 930 and the primary seal member 900 FIG. 1 when a device, such as an electrocautery instrument, is not inserted within central passage 600.

Referring now to FIG. 9, the secondary seal member 930 of the retaining clip 700 fits within a recess 710 defined by the distal stepped down portion 702 of the retaining clip 700. The second seal member 930 is of substantially the same thickness as the retaining clip recess 710. The secondary seal member 930 has one opening 940 for allowing the insertion of a laparoscopic tool such as an electrocautery device and may be made of any reasonably flexible material suitable for preventing leakage of bodily fluids, therethrough or therearound. Preferably, the retaining clip secondary seal 930 is fabricated of a rubber, or a plastic polymer of any known suitable composition, and the like. Referring back to FIG. 8, the retaining clip 700 includes a plurality of protrusions or nubs 750, which prevent the retaining clip 700 from rocking within the slot 800 of FIG. 6. By preventing the clip 700 from rocking within the slot 800 of FIG. 6, the secondary seal member 930 of the retaining clip 700 makes flush contact with the primary seal member 900 FIG. 3, enhancing the sealing effect.

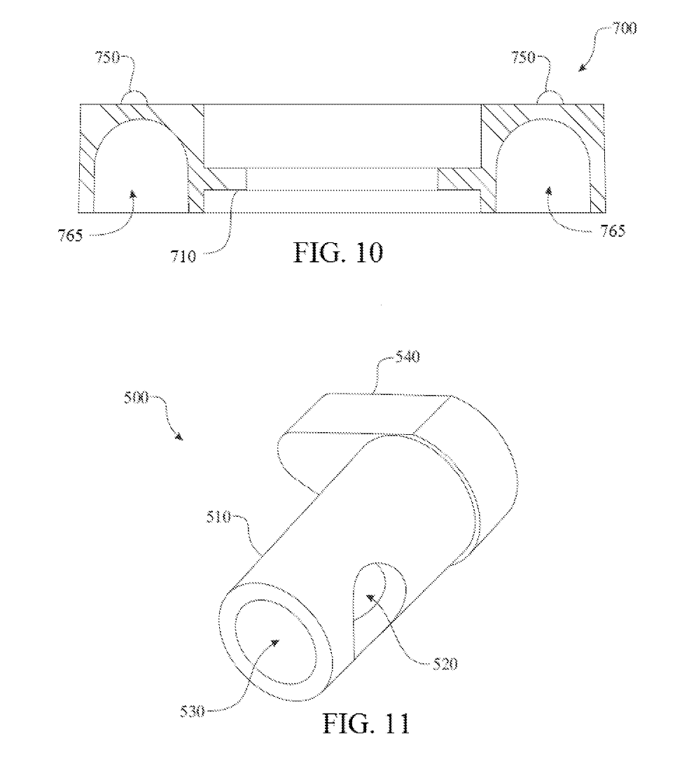

An end view of the retaining clip 700 is presented in FIG. 10. The retaining clip 700 includes small protrusions or nubs 750 for stabilizing the retaining clip 700 within the retaining clip slot 800, the nubs 750 engaging against the distal surface of the proximal wall 310. The retaining clip 700 includes recesses 765 for receiving biasing springs 770 which maintain shoulders 720 against the side walls 801 FIG. 7 of the retaining clip receiving slot 800, maintaining the retaining clip 700 in a normally closed position.

A hollow tube 510 defining an interiorized portion of the stopcock 500, extends generally perpendicularly to the handle 540 as illustrated in FIG. 11. The tube 510 includes two openings: a side opening 520 and a distal opening 530. The distal opening 530 is located at an end of the tube 510 that is opposite from handle 540.

The stopcock 500 is used to control the evacuation of smoke and/or bodily fluids from the operative site. All that is required for creating a fluid path from the operative site through vacuum attachment 300 is for opening 530 of stopcock 500 to be in fluid communication with vacuum connecting port 400 of FIG. 3.

The stopcock openings 520 and 530 allow for the selective passage of smoke, bodily fluids, and the like from the elongated tube 200, through the central passage 600, and through the stopcock 500. The hollow tube portion 510 allows the passage of smoke, bodily fluids, and the like through the stopcock 500 into the working hub 100 when the stopcock 500 is open. In the open position, the distal opening 520 of the stopcock 500 of FIG. 3 and is fluidly connected to the side bore 606 in the center tube 311 of FIG. 4. The path of fluid or smoke from the body to the vacuum source is as follows:

(1) Fluid enters the distal end 201 FIG. 1 of elongated tube 200 and travels through the tube 200 toward the working hub 100;
(2) at the side bore 606 in the center tube 311 FIG. 4, the fluid passes through a distal end opening 452 in the stopcock sleeve 450 and enters the central hollow passage defining the hollow tube portion 510 of stopcock 500;
(3) the fluid exits the stopcock 500 through a side opening 520 and a side opening 602 FIG. 7 in the stopcock sleeve 450;
(4) the fluid then travels through a bottom chamber 454 of working hub 100 defined between the center wall 328 FIG. 6 and the distal wall 320 and toward the interior open end 401 FIG. 4 of vacuum connecting port 400;
(5) the fluid exits the working hub 100 via vacuum port 400; and
(6) the fluid exits the working hub 100 through tubing 460 of FIG. 7 attached between the vacuum connecting port 400 and a vacuum source (not shown).

In FIG. 11 the closed position, the side opening 520 of stopcock 500 is not aligned with the opening 602 FIG. 7 in the stopcock sleeve 450 FIG. 4. Into which stopcock 500 enters via sidewall opening 513. Instead, a portion of the stopcock wall 515 covers the opening 602, preventing any vacuum draw.

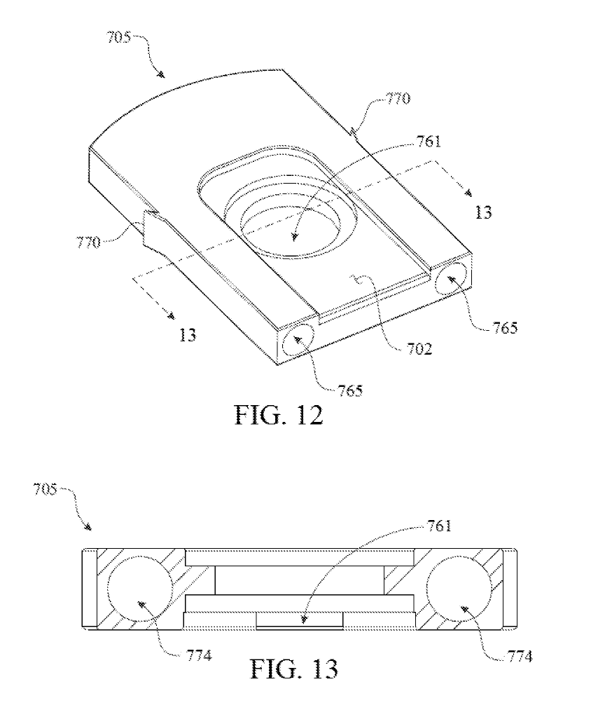

An alternative exemplary embodiment of a retaining clip 700, referred to as retaining clip 705, is presented in FIGS. 12 and 13. The alternative retaining clip 705 includes alternative quick connect protruding side tabs 770 which would releasably engage with cooperating grooves (not shown) within side walls 801 of the clip retaining slot 800 FIG. 6 of the working hub 100.

As shown in FIG. 14, the outer diameter of elongated hollow tube 200 is less than the inner diameter of standard cannula 2000. Thus, elongated tube 200 slidably fits inside a cannula 2000. In a preferred embodiment, cannula 2000 has an inner diameter of about 5 mm. The multifunction laparoscopic device 1000 is preferably sized to slide freely within cannula 2000. The multifunction laparoscopic device 1000 is preferably sized and configured so that any known embodiment of an electrocautery device or other laparoscopic instrument 3000 would pass through the central passage 600 of FIG. 1 and extend out beyond the distal end 201 of the multifunctional laparoscopic device 1000, with smoke and/or fluid evacuation occurring substantially at the smoke generating location. The multifunction laparoscopic device 1000 slides in the cannula 2000 without resistance. The multifunctional laparoscopic device 1000 is preferably constructed of a strong, lightweight, biocompatible material suitable for use during surgery, preferably of plastic, aluminum, stainless steel, or other rigid metal. A cannula 2000 may even be provided with a mechanism (not shown) which will cooperatively engage with the device 1000 so that surgeons may selectively position the device 1000 within the cannula 2000 and lock the device 1000 at the desired position within cannula 2000.

The inner diameter of the elongated hollow tube 200 is larger than the outer diameter of standard laparoscopic cautery device shafts or other laparoscopic tool shafts, such as laparoscopic tool 3000 shown in FIG. 14. The thin wall thickness of the elongated hollow tube 200 permits attachment of the multifunction laparoscopic device 1000 to an electrocautery device or laparoscopic tool and allows the device 3000 and the multifunction laparoscopic device 1000 to share the same cannula 2000. The sharing of cannula 2000 reduces the number of incisions in the patient and reduces surgery time.

The central passage 600 of the working hub 100 preferably includes two different diameter end openings such that the proximal end of the central passage 600 is larger than the distal end of the central passage 600. The resultant funnel shape 605 of FIG. 4 allows standard electrocautery and/or laparoscopic tools 3000 to be more easily inserted into the central passage 600, as shown in FIG. 14. The distal end of the central passage 600 is slightly smaller than the inner diameter of the elongated tube 200 to allow the elongate tube 200 to be permanently joined with the working hub 100.

In practice, the multifunction laparoscopic device 1000 vacuums smoke, fluids, and the like through an annular space between an inner diameter of the multifunction laparoscopic device 1000 and the shaft 3100 of an electrocautery device or laparoscopic tool 3000. Fluid is drawn toward vacuum connection port 400 where it exits the device. The vacuum/smoke evacuation function is controlled by operation of the stopcock 500. Rotation of the stopcock 500 controls the opening and closing of the passage 520, shown in FIG. 11, to a vacuum source (not shown), which is attached to vacuum port 400.

As shown in FIGS. 4, 5, and 14, retaining clip 700 serves as a sealing device for preventing pressurized CO₂ from exiting an insufflated abdomen (not shown). The position of multifunction laparoscopic device 1000 along the shaft of an electrocautery device or laparoscopic tool 3000, as shown in FIG. 14, is adjusted by moving multifunction laparoscopic device 1000 relative to said device shaft 3100. The retaining clip 700 may further include additional exemplary sealing structures to prevent loss of vacuum pressure therearound or therethrough.

Referring generally to FIG. 14, the multifunction laparoscopic device 1000 is shown seated about an electrocautery device 3000 during surgery. Electrocautery device 3000 is inserted into central passage 600 and into the elongated hollow tube 200 until a tip 3005 of the electrocautery device exits the distal end 201 of the elongated hollow tube 200. In a preferred embodiment, the overall length of multifunction laparoscopic tool 1000 is less than the shaft length of standard electrocautery devices 3000.

The retaining clip 700 must be engaged by pushing the retaining clip 700 completely into the slot 800 before an electrocautery or laparoscopic tool 3000 can enter the multifunction device 1000. For electrocautery devices 3000, the cauterizing tip 3005 is advanced down the elongated hollow tube 200 of the multifunction laparoscopic device 1000 until it exits the distal end 201 of the elongated hollow tube 200.

The vacuum port 400 is connected to a vacuum pump or source (not shown) via tube 460 shown in FIG. 7. The electrocautery device 3000 has a cauterizing tip 3005 which extends past the distal end 201 of the elongated hollow tube 200. Tissue within, for example, an abdomen, is brought into contact with the cauterizing tip 3005 to cauterize same. Smoke is generated when the cauterizing tip 3005 contacts bodily tissue. This smoke is removed from the abdomen via distal end 201 of elongated hollow tube 200.

In a proposed further exemplary embodiment, a sensing device may be operatively connected to the electrocautery device 3000 of the present invention which would automatically start smoke evacuation from the surgical site upon sensing activation of the electrocautery device 3000, blood coagulation, temperature, or time at the surgical site.

When the multifunction laparoscopic device 1000 suctions blood or other fluids from the surgical site, the orientation of the elongated hollow tube 200 relative to the shaft 3100 of the electrocautery or other laparoscopic device 3000 of FIG. 14 may be altered to improve suctioning. The surgeon will slide the multifunction laparoscopic device 1000 along the electrocautery or other laparoscopic tool shaft 3100 until the distal end 201 of the elongated hollow tube 200 extends past tip 3005 of the electrocautery device or laparoscopic tool 3000. Since the electrocautery or other laparoscopic tool is withdrawn within the elongated hollow tube 200 of the multifunction laparoscopic device 1000, the opening at the distal end 201 of the elongated hollow tube 200 can make direct contact with pools of blood and other fluids without interference from the tip of the electrocautery or laparoscopic device. Such orientation will enhance the suctioning of pools of blood or other fluids into the multifunction laparoscopic device 1000.

The multifunction laparoscopic device 1000 can also be operated as an irrigator during surgical procedures by replacing the vacuum source with a source of sterile fluid. The source of fluid can be an IV bag or any other common surgical fluid source. The fluid source can be manual; meaning gravity will cause the fluid to flow from the source to the multifunction laparoscopic device 1000, or the fluid may be pressurized by a pump (not shown). The source of fluid is connected to the vacuum port 400 of the multifunction laparoscopic device 1000 by means of tubing 460 capable of carrying fluid. In this instance, the surgeon uses the stopcock 500 to control in an on/off manner the provision of fluid to the operating site. However, it will be understood that fluid flow is reversed from the suctioning previously described. Fluid now flows from the vacuum port 400 through the working hub 100 before entering opening 452 FIG. 4 in the stopcock sleeve 450 and then through opening 520 in the stopcock 500. Once the fluid enters the stopcock 500, the fluid passes along the length of the stopcock 500 until it exits the stopcock 500 and enters side wall opening 606 into the center tube 311 of the central passage 600 and out the distal end 201 of elongated tube 200, reaching the operative site.

For increased efficiency, the suction and irrigation functions of the multifunctional laparoscopic device 1000 can share the same tubing, and valves external to the multifunctional laparoscopic device 1000 may be used to selectively connect either the vacuum source or the source of fluid to the multifunction laparoscopic device vacuum port 400. In another embodiment not shown an external automatic vacuum system and or device may be used to automatically evacuate the smoke filled air in the abdomen of the patient when cauterization or evacuation takes place.

The above-described embodiments are merely exemplary illustrations of implementations set forth for a clear understanding of the principles of the invention. Many variations, combinations, modifications, or equivalents may be substituted for elements thereof, some of which have been broached above, without departing from the scope of the invention. Therefore, it is intended that the invention not be limited to the particular embodiments disclosed as the best mode contemplated for carrying out the invention, but that the invention will include all embodiments falling within the scope of the appended claims.

What is claimed is:

1. A multifunctional surgical device for use during endoscopic surgery and particularly during electrocauterizing surgery for evacuating smoke and or fluid, or for providing fluids to the surgical area, the device comprising:
a working hub incorporating an elongated distally extending tube and having an inlet/outlet port to which a vacuum attachment and/or source of fluid is attached, said working hub comprising:
a side wall, a proximal wall, a distal wall and a center wall positioned between the proximal and distal walls;
the inlet/outlet port disposed through said side wall, said port being connectable to a source of vacuum and/or fluid;
a stopcock disposed through said side wall and being selectively, fluidly connected to said source of vacuum and/or fluid, such that said stopcock can allow or disallow a fluid connection between said port and said elongated tube; and
an open central passage disposed through said housing along said longitudinal axis;
the elongated tube having a proximal end and a distal end, said tube disposed coaxially with the axis of said central passage, said proximal end of said tube permanently joined with and forming a distal portion of said central passage.

2. The multifunctional surgical device of claim 1 wherein a portion of the device is sized and configured to be slidably received within a cannula and to extend past a distal tip of said cannula.

3. The multifunctional surgical device of claim 1 being sized and configured to slidably receive within the central passage thereof an endoscopic tool.

4. The multifunctional surgical device of claim 3 wherein the endoscopic tool is an electrocautery device.

5. The multifunctional surgical device of claim 3 further including a sealing mechanism for preventing loss of insufflation pressure from an insufflated abdomen of a patient when the endoscopic tool is withdrawn from the central passage of the device.

6. The multifunctional surgical device of claim 4 wherein the elongated tube has a distal end from which a distal end of the electrocautery device extends outwardly.

7. The multifunctional surgical device of claim 1 wherein the elongated tube has a proximal end frictionally engaged to a tube receiving stepped down nipple like structure within the working hub.

8. The multifunctional surgical device of claim 1 wherein the working hub is divided into two chambers by the center wall.

9. The multifunctional surgical device of claim 8 wherein one chamber comprises a proximal chamber.

10. The multifunctional surgical device of claim 8 wherein another chamber comprises a distal chamber.

11. The multifunctional surgical device of claim 10 wherein the vacuum port engages through the side wall of the distal chamber.

12. The multifunctional surgical device of claim 10 wherein a hollow stopcock engaging sleeve is positioned within the distal chamber.

13. The multifunctional surgical device of claim 12 wherein the hollow stopcock engaging sleeve includes an open distal end and an opening in a side wall thereof, the opening in the side wall being positioned within the distal chamber.

14. The multifunctional surgical device of claim 12 wherein the elongated tube has an opening in the wall thereof within the distal chamber which fluidly engages the open distal end of the hollow stopcock engaging sleeve.

15. The multifunctional surgical device of claim 14 wherein the stopcock includes a hollow bore therein and wherein a side wall of the stopcock includes a side opening therein which is selectively capable of being manually aligned with the side opening in the stopcock sleeve or being moved out of alignment therewith.

16. The multifunctional surgical device of claim 15 wherein material being vacuumed from the site of the endoscopic surgery enters the distal end of the elongated tube, travels to the side opening in the elongated tube and into and through the stopcock, passes through the side opening in the stopcock and the side opening in the stopcock sleeve when the openings are aligned, passes across the distal chamber and exits through the port in the side wall of the distal chamber.

17. The multifunctional surgical device of claim 15 wherein fluid material being provided to the site of the endoscopic surgery enters through the port in the side wall of the distal chamber, passes across the distal chamber, enters the stopcock through the side opening in the stopcock sleeve and the side opening in the stopcock when the side openings are aligned, passes through the stopcock to the side opening in the elongated tube and flows out of the open distal end of the elongated tube.

18. The multifunctional surgical device of claim 1 further includes means for closing off the central passage when no tool is present therein so that insufflation of the body cavity is not lost.

19. The multifunctional surgical device of claim 18 wherein the means for closing off the central passage comprise a retaining clip which is manually moveable from an open position to a closed position.

20. The multifunctional surgical device of claim 18 wherein the means for closing off the central passage comprise a retaining clip which is spring biased.

21. A multifunctional surgical device comprising:
a working hub having an elongated tube extending distally therefrom;
the working hub defining a fluid cavity therein which engages the elongated tube to a source of fluid or vacuum.

22. A multifunctional surgical device for use during surgery comprising:
a working hub having a central passage and an inlet/outlet port to which a vacuum attachment and/or source of fluid is attached; and
an elongated tube having a proximal end and a distal end and being disposed coaxially with said central passage, said proximal end being attached to said working hub and extending therefrom, said distal end of said elongated tube being sized for insertion into a bodily opening;
wherein the multifunctional surgical device evacuates smoke and/or fluid, or provides fluids to the surgical area through said vacuum attachment and/or source of fluid, respectively.

23. The multifunctional surgical device of claim 22 wherein said central passage of said working hub is sized for accepting a surgical tool therein.

24. The multifunctional surgical device of claim 23 configured to allow said surgical tool to pass through said central passage of said working hub and through said elongated tube to extend past the distal end of said elongated tube to access the working area within the body.

25. The multifunctional surgical device of claim 24 wherein the device evacuates smoke and/or fluid, or provides fluids to the surgical area through said vacuum attachment and/or source of fluid, respectively, through said elongated tube while the surgical tool is inserted therethrough.

26. The multifunctional surgical device of claim 22 further comprising a sensing device.
